(19) United States
(12) Patent Application Publication
(10) Pub. No.: US 2011/0159074 A1
(43) Pub. Date: Jun. 30, 2011

(54) Wipe Article Comprising Lotion Composition Comprising Omega-6 Fatty Acid And Method Of Improving Skin Barrier Function

(76) Inventors: Raphael Warren, Amberley Village, OH (US); Thomas James Klofta, Cincinnati, OH (US); Randall Glenn Marsh, Hamilton, OH (US)

(21) Appl. No.: 12/976,180

(22) Filed: Dec. 22, 2010

Related U.S. Application Data
(63) Continuation-in-part of application No. 12/974,731, filed on Dec. 21, 2010.
Application No. 12/974,731 is a continuation-in-part of application No. 12/974,674, filed on Dec. 21, 2010.
(60) Provisional application No. 61/291,069, filed on Dec. 30, 2009.

(51) Int. Cl. A61K 9/70; A61K 31/201; A61K 31/202; A61K 31/232; A61K 31/231; A61K 36/31; A61K 36/185; A61P 17/00; A61P 17/16

(52) U.S. Cl. 424/443; 514/560; 514/547; 514/549; 424/725

(57) ABSTRACT

A wipe article comprising a composition comprising omega-6 fatty acid. A method of improving skin barrier function of semi-occluded skin comprising the step of contacting the semi-occluded skin with a wipe comprising an omega-6 fatty acid.

### CROSS REFERENCE TO RELATED APPLICATION

This application is a continuation-in-part of U.S. patent application Ser. No. 12/974,674, filed Dec. 21, 2010, and a continuation-in-part of U.S. patent application Ser. No. 12/974,731, filed Dec. 21, 2010, each of which claims the benefit of Provisional U.S. Patent Application Ser. No. 61/291,069, filed Dec. 30, 2009. All three of these applications are hereby incorporated herein by reference in their entireties.

### FIELD OF THE INVENTION

The present invention relates to a wipe article comprising a lotion composition comprising omega-6 fatty acid and a method of improving skin health by contacting the skin with the wipe article.

### BACKGROUND OF THE INVENTION

Disposable absorbent articles, such as diapers, training pants, and catamenial devices having lotioned topsheets are known. Lotions of various types are known to provide various skin benefits, such as prevention or treatment of diaper rash. These lotions can be applied to the topsheet of absorbent articles, for example, and can be transferred to the skin of the wearer during use. Similarly, wipe articles containing lotions are known.

Many types of disposable absorbent products, such as diapers, are available that have a high capacity for absorbing urine and other body exudates. Disposable products of this type generally comprise some sort of liquid-pervious topsheet material, an absorbent core, and a liquid-impervious backsheet material. Although these types of absorbent structures may be highly efficient for the absorption of liquids, it is well recognized that long-term wear of such structures may lead to skin which is compromised in terms of being over hydrated or exposed to skin irritants commonly found in body exudates. It is generally known that skin under absorbent articles is more susceptible to skin disorders, including diaper rash, erythema (i.e., redness), heat rash, abrasion, pressure marks and skin barrier loss. For example, 21 C.F.R. 333.503 defines diaper rash as “[a]n inflammatory skin condition in the diaper area (perineum, buttocks, lower abdomen, and inner thighs) caused by one or more of the following factors: moisture, occlusion, chafing, continued contact with urine or feces or both, or mechanical or chemical irritation.”

To address the concerns of skin disorders associated with wearing absorbent articles, the caregiver often applies skin protective products such as Vaseline®, medicated ointments, powders, etc. to the buttocks, genitals, anal and/or other regions before placing the absorbent article on the wearer. This procedure usually involves the caregiver applying the skin protective product to the caregiver's hands, and then wiping the same on the skin of the infant. To eliminate the need for this wasteful, messy, time-consuming, and easily forgotten procedure, there have been attempts to prepare absorbent articles which contain a protective or therapeutic skin care substance on the article's topsheet.

Other approaches to ameliorating diaper rash have included the addition of protective or therapeutic skin care substances to the lotion of a baby care wipe. The protective or therapeutic skin care substances may transfer to a baby's skin during a diaper change, when the baby care wipe is wiped across the surface of the skin, as to clean and care for the skin.

Despite these improvements, diaper rash remains a problem. The website e-medicine.com, in an article titled “Pediatrics, Diaper Rash” and dated Mar. 17, 2010, reported that diaper rash is the most common dermatitis found in infancy. Prevalence has been variably reported from 4-35% in the first 2 years of life, and incidence triples in babies with diarrhea. It is not unusual for every child to have at least 1 episode of diaper rash by the time he or she is toilet-trained.

Accordingly there is a continuing desire for a wipe article that reduces the discomfort associated with wearing absorbent articles and can improve the skin health and/or skin barrier function of skin at high risk for diaper rash.

### SUMMARY OF THE INVENTION

In some aspects, this disclosure relates to a wipe comprising a substrate and an oil material. The oil material may comprise and omega-6 fatty acid having an OSI of at least about 10 hours.

In some aspects, this disclosure relates to a method of improving skin barrier function of semi-occluded skin. The method may comprise contacting the semi-occluded skin with a wipe comprising a substrate and an oil material. The oil material may comprise an omega-6 fatty acid having an OSI of at least about 10 hours.

### DETAILED DESCRIPTION OF THE INVENTION

As used herein, the term “fatty acid” refers to fatty acid moieties in a triglyceride as well as “free” fatty acid molecules within a given oil. A triglyceride is a glycerin molecule esterified with three fatty acid groups. Many refined oils include both triglycerides containing three fatty acid moieties, and at least trace amounts (more than 0%, but less than 1%) of free fatty acids that are not part of a triglyceride molecule. The fatty acid moieties of a particular triglyceride molecule may be the same or different.

As used herein, the term “wipe article” refers to a piece of material, generally non-woven material, used to cleanse body parts. Some currently available wipe articles are intended for cleaning of the peri-anal area after defecation. Other wipe articles are available for cleaning the face or other body parts. Wet-wipe articles may comprise an aqueous lotion. The aqueous lotion may itself facilitate cleaning (as by dissolving soils), or may serve as a carrier for other active compounds, such as surfactants, emollients, skin protectants, and such. Wipe articles may comprise an anhydrous coating. The anhydrous coating, like an aqueous lotion, may itself facilitate cleaning (as by dissolving non-polar soils), or may serve as a carrier for other active compounds, such as surfactants, emollients, skin protectants, and such. Some wipe articles may comprise both an aqueous lotion and an anhydrous coating.

Wipe articles are generally of sufficient dimension to allow for convenient handling while being small enough to be easily disposed of by the sewage system or discretely disposed of in garbage bins. The material, or substrate, of the wipe articles is generally soft and flexible, potentially having a structured surface enhancing its cleaning performance. The substrate may be a non-woven material, generally made of synthetic compounds. However, woven materials as well as the use of natural compounds in either woven or nonwoven materials are within the scope of the present disclosure. The texture and material of the wipe article may be relevant to the performance of the wipe article. Some exemplary non-woven materials comprise fibers selected from the groups consisting of polyolefins, polyesters, celluloses, rayons, polyamides, polyesteramides, polyvinyl alcohols, and combinations thereof. A substrate may be manufactured via any process, such as, but not limited to, spunlace, meltblowing, airlaying, coform, and carding processes, and may have a dry basis weight of between about 25 grams per square meter (gsm) and 75 gsm, or between 30 gsm and 65 gsm. Other substrates may be used with the lotion and/or coating compositions described below.

The size of the wipe article can vary. The wipe article can be greater than or equal to about 4 square inches (about 25 square centimeters) in size, greater than or equal to about 9 square inches (about 50 square centimeters) in size, less than or equal to about 225 square inches (about 1,450 square centimeters) in size, between about 16 square inches (about 100 square centimeters) and about 50 square inches (about 320 square centimeters), or about 35 square inches (about 225 square centimeters) in size. Typically, each individual wipe is arranged in a folded configuration and stacked one on top of the other to provide a stack of wipes. Such folded configurations include c-folded, z-folded, quarter-folded configurations and the like. The folded wipes may be interleaved. The stack of folded wipes may be placed in the interior of a container, such as a plastic tub or flexible refill bag, to provide a package of wipes for eventual sale to the consumer. Interleaved wipes may be useful for “pop-up dispensing,” where pulling a first wipe through an opening in a container also pulls the next wipe in the stack at least partially into or through the opening. In this way, it is easy to grasp and pull each successive wipe during dispensing. Alternatively, the wipes may include a continuous strip of material which has perforations between each wipe and which may be arranged in a stack or wound into a roll for dispensing.

The wipe article may be a cleansing wipe. The wipe article can also be a hygienic cleansing wipe, such as a baby wipe, that may be used by the wearer to clean feces and/or other body exudates from the body. Of course, a wipe intended for use as a baby wipe may be useful for cleaning other body parts or surfaces, on the baby or elsewhere. For example, a baby wipe may also be used to clean food or other soils from the body of a baby, toddler, child, or adult, or to clean menses, sweat, or other exudates or soils from the body of an adult. The cleaning of menses may be important because when menses leaves the wearer's body, it may tend to smear over the pudendal region of the wearer's body and be retained on the wearer's skin and pubic hair. Furthermore, menses, like feces, may then dry on the skin and/or in the pubic hair, and make later cleansing difficult.

Without being bound by theory, it is believed that a textured wipe may further enable the ease of removal of the bodily exudates by improving the ability to grip or otherwise lift the exudates from the skin during cleansing. Any one of a number of texture elements may be useful in improving the ability to grip or otherwise lift the exudates from the skin during cleansing such as, but not limited to continuous hydro-molded elements, hollow molded element, solid molded elements, circles, squares, rectangles, ovals, ellipses, irregular circles, swirls, curly cues, cross hatches, pebbles, lined circles, linked irregular circles, half circles, wavy lines, bubble lines, puzzles, leaves, outlined leaves, plates, connected circles, changing curves, dots, honeycombs, etc. and combinations thereof. The texture elements may be hollow elements. The texture elements may be connected to each other. The texture elements may overlap each other. Exemplary texture elements are described, for example, in U.S. Patent Application Publication 2007/0254145 and U.S. Pat. No. 7,771,648.

Wet-wipe articles may be impregnated with a liquid, semi liquid, semi solid, or solid composition. A semi liquid composition comprises primarily—50% or more by weight—of liquid components, but also comprises some solid components. A semi solid composition comprises primarily—50% or more by weight—of solid components, but also comprises some liquid components. The compositions may be called a lotion (if aqueous or hydrophilic) or a coating (if anhydrous). The lotion and/or coating may enhance cleaning, provide a smooth and soft feeling, care for the skin, or provide all of these benefits. Generally a lotion composition is of sufficiently low viscosity to impregnate the entire structure of the wipe article. In some other instances, the composition can be primarily present at the wipe article surface and to a lesser extent in the inner structure of the wipe article. In one embodiment the composition is releasably carried by the substrate, that is, the composition is contained either in or on a substrate and is readily releasable from the substrate by applying some force to the substrate, for example, wringing the substrate, or wiping a surface, such as a child's bottom, with the wet-wipe article. Alternatively, the wipe may also be dried after it is loaded with the lotion and/or coating, creating a dry wipe. Prior to use, water may be added to a dry wipe to increase the amount of lotion and/or coating released.

It is generally believed that the integrity of the skin barrier is related to the lipid composition of the stratum corneum, e.g., ceramides, cholesterol, triglycerides, cholesterol esters, etc. As used herein, the term “lipid” includes, but is not limited to, fats and oils and their acid analogues (i.e., fatty acids). Compositional changes, in particular to the ceramide class of lipids, feature prominently as a consequence of genetic predisposition (i.e., atopy, X-linked ichthyosis), environmental insults (i.e., irritants, winter cold/dry skin or xerosis, UV-light), or disease (i.e., psoriasis). Ceramide EOS (formerly identified as ceramide 1) has been linked with skin barrier functionality. For example, ceramide EOS may be cross-linked to the protein envelope of the corneocyte and is believed to be the molecular ‘rivet’ that organizes the lipid lamellae. This ceramide link has led to development of topical remedies that contain lipid supplements (i.e., ceramides or ceramide derivatives) thought to be corrective for interior skin harrier activity. Despite a link between lipid composition and skin barrier, little is known about the skin surface lipid composition of semi-occluded skin, such as skin that is more-or-less continually covered by an absorbent article, like a baby's buttocks, genitals, and intertriginous regions of the thighs and buttocks. Despite considerable differences in skin barrier behavior, recent reports show there may be little to no meaningful difference between the superficial lipid composition of semi-occluded skin and non-occluded skin. Indeed, data suggest that a more important factor contributing to skin barrier properties is the relative abundance of the unsaturated fatty acid that is esterified to the sphingosine base of ceramide POS. For example, the greater the amount of linoleate, the more intact is the skin barrier. Data suggest that despite no difference in the relative abundance of ceramide EOS between semi-occluded and non-occluded skin, there is a 75% reduction in the abundance of omega-6 fatty acid ester linoleate in semi-occluded skin relative to non-occluded skin.

An interesting approach to remedy the inferior semi-occluded skin barrier, and thus improve skin comfort, would be to supplement the semi-occluded skin, or portions thereof, with compositions rich in essential fatty acids or lipids, in particular, omega-6 fatty acids or fatty acid esters. As used herein, the term “essential fatty acids” means fatty acids which cannot be synthesized by the human body and must be obtained from a dietary source. Because humans lack the required enzyme to introduce carbon-carbon double bonds at carbon atoms beyond the ninth carbon atom in unsaturated fatty acids (the ninth carbon atom from the omega end of the chain), linoleic acid (an omega-6 fatty acid) and alpha linolenic acid (an omega-3 fatty acid) are essential fatty acids that must be obtained by humans from a dietary source to ensure good wellness. Many humans have been found to be deficient in essential fatty acids which can lead to numerous heath ailments and problems. It is well known that common oils such as those derived from the sunflower, low erucic rapeseed (or canola), flax (or linseed), soybean, etc. contain an abundance of unsaturated essential fatty acids, including the desirable omega-6 fatty acids. Dietary supplementation with materials rich in essential fatty acids (e.g., flax seed oil, fish oil, borage oil, evening primrose oil) are believed to ameliorate skin ailments. However, dietary supplementation requiring large doses (gram per day) can be wasteful, and the supplemental oils do not necessarily reach the semi-occluded skin target.

To address the deficiency in omega-6 fatty acid ester linoleate in semi-occluded skin, a wipe article may comprise a lotion and/or coating composition comprising omega-6 fatty acid. The lotion and/or coating composition may comprise at least about 0.003%, from about 0.003% to about 35%, from about 0.015% to about 25%, or from about 0.06% to about 20%, by weight of the lotion and/or coating composition, of omega-6 fatty acid.

The omega-6 fatty acid may be added to the lotion and/or coating as an oil material, such as from a vegetable oil. Therefore, in one embodiment, the lotion and/or coating composition comprises an oil material comprising omega-6 fatty acid. The lotion and/or coating composition may comprise from about 0.1% to about 70%, from about 0.5% to about 50%, or from about 2% to about 40%, by weight of the lotion and/or coating composition, of the oil material. The oil material may comprise at least 3%, from about 3% to about 50%, or from about 5% to about 40%, by weight of the oil material, of omega-6 fatty acid.

It is, however, well known that unsaturated fatty acids are not stable and easily oxidize. Oxidation can be promoted by multiple sources, including temperature, light, air, oxygen, moisture, and metals (in particular copper). Product instability may be influenced by the lotion making and application process. For example, melting and mixing the lotion ingredients can require high temperatures (above the melting point of the ingredients, e.g., greater than 70° C.). After making and before application of the lotion and/or coating to a substrate, the lotion may be stored in a tank for a considerable time (e.g., >24 hr), Another source of instability is the shelf storage of the finished product. It is not unusual for product to remain on the shelf (in the store and/or at home) for at least a year and, depending on geographical location, storage temperatures can exceed 40° C. Water or glycol-based lotions may be more susceptible to instability than oil-based lotions and/or coating, due to dissolved oxygen in the water or glycol. Collectively, these factors can lead to oxidation and creation of reactive oxygen free radicals or active oxygen. This can lead to product deterioration such as discoloration (i.e., yellowing) and rancid odor.

A common measure for monitoring oxidative stability is the development of hydroperoxides (peroxide value or PV) over time. Oxidative stability can also be expressed in terms of the time required to obtain secondary oxidation products when aerating a sample at elevated temperature. A suitable measure of oxidative stability is called the Oil Stability Index (referred to herein as “OSI”). The OSI of an oil material can be measured according to the American Oil Chemical Society Oil Stability Index Method (AOCS Official Method Cd 12b-92).

In some embodiments, the oil material is selected to have an oil stability index (“OSI”) of at least about 10 hours, at least about 14 hours, or at least about 18 hours.

It is believed that oil materials comprising relatively high levels of oleic fatty acid tend to be more stable in the context of a wet-wipe product. In one embodiment, the oil material comprises at least about 10%, from about 10% to about 80%, or from about 15% to about 70%, by weight of the oil material, of oleic fatty acid. In one embodiment, the lotion and/or coating composition comprises from about 0.01% to about 56%, from about 0.05% to about 40%, or from about 0.2% to about 32%, by weight of the lotion and/or coating composition, of oleic fatty acid.

It is believed that oil materials comprising relatively low levels of linolenic fatty acid (omega-3 fatty acid) tend to be more stable in the context of a wet-wipe product. In one embodiment, the oil material comprises less than about 10%, from about 10% to about 5%, or from about 5% to about 0%, by weight of the oil material, of linolenic fatty acid. In one embodiment, the lotion and/or coating composition comprises from about 7% to about 0%, from about 5% to about 0%, or from about 4% to about 0%, by weight of the lotion and/or coating composition, of linolenic fatty acid.

Non-limiting examples of suitable oil materials exhibiting the desired properties described herein include high oleic canola Oil (Brassica campestris, B. napus, B. rapa; characterized by having an oleic fatty acid content greater than 70%, e.g., high oleic canola oil, very high oleic canola oil, or partially hydrogenated canola oil), manrula kernel oil (Sclerocarya birrea), palm oil (Elaeis Guineensis Oil), palm olein, palm stearin, palm superolein, pecan oil, pumpkin seed oil, oleic safflower oil (Carthamus Tinctorius; characterized by having an oleic fatty acid content of greater than about 30% and omega-6 fatty acid content of less than about 50%, e.g., high oleic safflower oil), sesame oil (Sesamum indicum, S. oreintale), soybean oil (Glycine max, e.g., high oleic soybean, low linolenic soybean oil, partially hydrogenated), high oleic sunflower oil (Helianthus annus; characterized by having an oleic content of greater than about 40%, e.g., mid oleic sunflower or high oleic sunflower oil), and mixtures thereof. Oleic canola oil, palm oil, sesame oil, high oleic safflower oil, high oleic soybean oil, mid oleic sunflower oil, and high oleic sunflower oil are common plant-bred derived oils and may be also be derived from non-genetically modified organisms (non-GMO).

Nonlimiting examples of oil materials are commercially-available from a number of vendors, including Cargill for partially hydrogenated soybean oil (i.e., Preference® 110W Soybean Oil or Preference® 300 Hi Stability Soybean Oil), mid oleic sunflower oil (i.e., NuSun® Mid-Oleic Sunflower Oil), high oleic sunflower oil (i.e., Clear Valley® High Oleic Sunflower Oil), high oleic canola oil, very high oleic canola, and partially hydrogenated low erucic rapeseed oil (i.e., Clear Valley® 65 High Oleic Canola Oil and Clear Valley® 75 High Oleic Canola Oil); Lambert Technology for high oleic canola oil (i.e., Oleocal C104); Arch Personal Care for manila kernel oil; Pioneer for high oleic soybean oil (i.e., Plenish®); Asoyia for low linolenic soybean oil (i.e., Ultra Low Linolenic Soybean Oil®); and Dipasa, Inc. for refined sesame oil.

It should be noted that the grade of oil material can be important as well in achieving the desired properties of the oil material as described herein. For example, the source of the oil material can be important, as the same oil (e.g. sesame oil) can exhibit a wide range of OSI values depending upon the source of the oil material. Similarly, the refining technique and processing conditions can influence the OSI of the oil material.

The oil material can further comprise a blend of oils, including those described supra, as well as additional oil materials. Suitable additional oil materials can include acai berry oil, almond oil, avocado oil, beech oil, brazil nut oil, camelina sativa oil (family Brassicaceae, e.g. Camelina Sativa, Gold of Pleasure, False Flax, etc.), camellia seed oil, canola oil, carrot seed oil, cashew nut oil, caster oil, cherry kernel oil, chia oil, corn oil, cottonseed oil, hydrogenated cottonseed oil, evening primrose oil, filbert (hazelnut) oil, grapeseed oil, hemp oil, hickory nut oil, jojoba oil, kukui oil, lanolin, olive oil (Olea europaea), macadamia oil, maring a oil, meadowfoam oil, neem oil, palm kernel oil, olive oil, passionflower oil (family Passiflora, Passiflora Incarnata), peanut oil, peach kernel oil, pistachio nut oil, rapeseed oil, rice bran oil, rose hip oil, safflower oil, sorghum oil, soybean oil, sunflower seed oil, tall oil, vegetable oil, vegetable squalene, walnut oil, wheat germ oil, and mixtures thereof. The oil material of the present invention can be selected from the group consisting of camelina sativa seed oil, oleic canola oil, evening primrose oil, manila kernel oil, palm oil, palm olein, palm stearin, palm superolein, passiflora incarnata seed oil, pecan oil, pumpkin seed oil, oleic safflower oil, sesame oil, soybean oil, oleic sunflower oil, vegetable oil, and mixtures thereof.

Suitable, commercially available oil materials include a mixture of vegetable oil and camelina sativa seed oil (commercially-available as Lipex® Omega 3/6 from Aarhus Karishamn Sweden AB), a mixture of vegetable oil and passiflora incarnata seed oil (commercially-available as Lipex® Omega Passiflora from Aarhus Karlshamn Sweden AB), a mixture of vegetable oil and evening primrose oil (commercially-available as Lipex Omega EPO from Aarhus Karishamn Sweden AB), high oleic canola oil (commercially-available as Clear Valley® 75 High Oleic Canola Oil from Cargill), and mixtures thereof.

To further enhance the stability of the oil material, certain antioxidants can be added to the oil or to the lotion and/or coating composition. In some embodiments, the oil material comprises from about 0.005% to about 1%, from about 0.01% to about 0.5%, or from about 0.02% to about 0.2%, by weight of the oil material, of an antioxidant. In some embodiments, the lotion and/or coating composition comprises from about 0.01% to about 1%, from about 0.05% to about 0.75%, or from about 0.2% to about 0.6%, by weight of the lotion and/or coating composition, of an antioxidant. Non-limiting examples of suitable antioxidants include Vitamin E (tocopherol, including α-tocopherol, β-tocopherol, γ-tocopherol, and δ-tocopherol), tocotrienol, rosemary, oil of rosemary, ascorbic acid, sesamol, sesamolin, sesamin, catechin, citric acid, tocopherol acetate, naringenin, and mixtures thereof.

In some embodiments, the oil material may be inherently high in antioxidants, such that the oil and/or the lotion and/or coating do not require supplementation with additional antioxidants. Exemplary, high-antioxidant oils include refined canola oil available as Lipex® Bassol C, and refined sunflower oil available as Akosun, both commercially available from Aarhuskarlsharmn Sweden AB, of Karlshamn, Sweden. It should be understood that the extraction and/or refining processes used to manufacture a given oil may alter the antioxidant concentration of the final oil, and that not all oils of the same type (e.g., canola) will have the same antioxidant concentration.

### Anhydrous Coatings

In some embodiments, the wet-wipe product may comprise an anhydrous coating, that is, a coating comprising less than about 40%, or less than about 20%, or less than about 5% water, by weight of the water to weight of the coating. The coating composition may further comprise a carrier. The carrier can help to deliver the omega-6 fatty acid to the skin of the user of the wipe article. The carrier can be included in the compositions as an individual carrier or a combination of carrier ingredients. The carrier can be a liquid, solid, or semisolid carrier material (at 30° C.), or a combination of these materials, and preferably forms a homogenous mixture or solution at selected processing temperatures for the resultant omega-6 fatty acid. Processing temperatures for the carrier system typically do not exceed 90° C., and may be held as low as possible to protect the omega-6 fatty acids, and other ingredients, from oxidation or decomposition. The carrier may comprise from 60 to 99.9%, or from 70 to 98%, or from 80 to 97%, by weight, of the coating. Exemplary anhydrous coating carriers include petrolatum, fatty alcohols, and natural and synthetic waxes, as described in further detail below.

Exemplary carrier compounds include petroleum-based hydrocarbons having from about 4 to about 32 carbon atoms, fatty alcohols having from about 12 to about 24 carbon atoms, polysiloxane compounds, fatty acid esters, alkyl ethoxylates, lower alcohols having from about 1 to about 6 carbon atoms, low molecular weight glycols and polyols, fatty alcohol ethers having from about 12 to about 28 carbon atoms in their fatty chain, lanolin and its derivatives, glyceride and its derivatives including acetoglycerides and ethoxylated glycerides of C12-C28 fatty acids, natural and synthetic waxes, and mixtures thereof.

Other carriers suitable herein can include oils or fats such as natural oils or fats, or natural oil or fat derivatives, in particular of plant or animal origin. Non-limiting examples include apricot oil, babassu oil, castor oil, coconut oil, cod liver oil, hydrogenated corn oil, hydrogenated cottonseed oil, hazelnut oil, jojoba oil, macadamia oil, meadowfoam seed oil, mink oil, maring a oil, manila oil, mortierella oil, palm kernel oil, hydrogenated peanut oil, hydrogenated rapeseed oil, rose hip oil, hydrogenated safflower oil, hydrogenated soybean oil, hydrogenated sunflower oil, hydrogenated walnut oil, hydrogenated wheat germ oil, or the hardened derivatives thereof, and mixtures thereof.

Suitable carriers further encompass waxes. As used herein, the term ‘wax’ refers to oil soluble materials that have a waxy constituency and have a melting point or range of above ambient temperature, in particular above 25° C. Waxes are materials that have a solid to semi-solid (creamy) consistency, crystalline or not, being of relative low viscosity a little above their liquefying point. Suitable waxes which can be incorporated into the lotion composition include animal, vegetable, mineral or silicone based waxes which may be natural or synthetic, and including mixtures thereof. Waxes can include, but are not limited to, natural waxes of vegetal origin, such as bayberry wax, candelilla wax, carnauba, ceresin, shea butter, cocoa butter. Japan wax, jojoba wax, ouricury wax, montan wax, rice bran wax, stearyl dimethicone, fruit-derived waxes, such as orange wax, lemon wax, and the like; and waxes from animal origin such as beeswax, woolwax, bear fat, whale blubber, walrus fat, lanolin wax, mink wax, and the like. Natural waxes further comprise mineral waxes such as ceresin and ozokerite waxes. Synthetic waxes include petroleum-based waxes, such as paraffin, and microcrystalline wax. Further suitable synthetic waxes are polyalkylene and polyethyleneglycol waxes, e.g. polyethylene wax; waxes based on chlorinated naphtalenes such as ‘Halowax’, synthetic hydrocarbon waxes, and the like, PEG-6 beeswax, PEG-8 beeswax, C30 alkyl dimethicone, synthetic beeswax, synthetic candelilla wax, synthetic camuba wax, synthetic ceresin wax, synthetic ozokerite wax, synthetic japan wax, synthetic jojoba wax, motan acid wax, motan wax, ouricury wax, rezowax, including mixtures thereof. Combinations of different kinds of waxes may be used.

Other suitable carriers include materials that act as solidifying agents, including some of the materials described hereinbefore. Suitable solidifying agent(s) in the coating compositions of the present invention can function to help solidify the composition so that the composition is a solid at room temperature and has a melting point of at least 30° C. The solidifying agent may also provide a tackiness to the composition that improves the transfer by adhesion to the skin of the wearer. Depending on the solidifying agent selected, the solidifying agent can also modify the mode of transfer so that the composition tends to fracture or flake off instead of actually rubbing off onto the skin of the wearer which can lead to improved transfer to the skin. The solidifying agent may further function as an emollient, occlusive agent, moisturizer, barrier enhancer, viscosity enhancer, and combinations thereof. Suitable solidifying agents include alkyl siloxanes, polymers like ethylene vinyl acetate, hydrogenated vegetable oils having a melting point of 35° C. or greater, fatty acid esters with a melting point of 35° C. or greater, alkyl hydroxystearates, branched esters, alkoxylated alcohols, fatty alcohols having a melting point of 35° C. or greater, natural and synthetic waxes with a melting point of 35° C. or greater, and alkoxylated carboxylic acid.

Additionally, or alternatively, the solidifying agents can be selected from animal, vegetable and mineral waxes and alkyl silicones. Examples include, but are not limited to, alkyl silicones, alkyl trimethylsilanes, beeswax, behenyl behenate, behenyl benzoate, C24-C28 alkyl dimethicone, C30 alkyl dimethicone, cetyl methicone, stearyl methicone, cetyl dimethicone, stearyl dimethicone, cerotyl dimethicone, candelilla wax, camuba wax, synthetic carnuba wax, PEG-12 carnauba, ceresin wax, hydrogenated microcrystalline wax, jojoba wax, microcrystalline wax, lanolin wax, ozokerite, paraffin wax, synthetic paraffin wax, cetyl esters, behenyl behenate, C20-C40 alkyl behenate, C2-C5 lactate, cetyl palmitate, stearyl palmitate, isostearyl behenate, lauryl behenate, stearyl benzoate, behenyl isostearate, cetyl myristate, cetyl octanoate, cetyl oleate, cetyl ricinoleate, cetyl stearate, decyl oleate, di C2-C5 alkyl fumerate, dibehenyl fumerate, myristyl lactate, myristyl lignocerate, myristyl myristate, myristyl, stearate, lauryl stearate, octyidodecyl stearate, octyidodecyl stearoyl stearate, oleyl arachidate, oleyl stearate, tridecyl behenate, tridecyl stearate, tridecyl stearoyl stearate, pentaerythrityl tetrabehenate, penteerythritylhydrogenated rosinate, pentaerythrityl distearate, peritaerythrityltetraabeite, penteerythrityl tetracocoate, penteerythrityl tetraperiargonate, pentserythrityl tetrastearate, ethylene vinyl acetate, polyethylene, hydrogenated cottonseed oil, hydrogenated vegetable oil, hydrogenated squalene, hydrogenated coconut oil, hydrogenated jojoba oil, hydrogenated palm oil, hydrogenated palm kernel oil, hydrogenated olive oil, polyamides, metal stearates and other metal soaps, C30-C60 fatty alcohols, fatty amides of 20 carbons or more, polypropylene, polystyrene, polybutane, polybutylene terephthalate, polydipentane, polypropylene, zinc stearate, dodecyl laurate, stearyl palmitate, octadecyl hexedecanoate, octadecyl palmitate, stearyl behenate, docosyl octanoate, tetradecyl-octadecanyl behenate, hexadecyl-cosanyl hexacosanate, shellac wax, glycol montanate, fluorinated waxes, C20-C40 alkyl hydroxystearyl stearate, and mixtures of such compounds.

### Aqueous Lotion Compositions

In some embodiments, the wet-wipe product may comprise an aqueous lotion, that is, a lotion comprising at least 50%, or at least 75%, or at least 90% water, by weight of the water to weight of the lotion. An emulsifier may be used to incorporate an omega-6 fatty acid in an aqueous (oil-in-water emulsion) lotion. Exemplary emulsifiers include, but are not limited to, laureth-23; ceteth-2; ceteth-10; ceteth-20; ceteth-21; ceteareth-20; steareth-2; steareth-10; steareth-20; oleth-2; oleth-10; oleth-20; steareth-100; steareth-21; PEG-40 sorbitan peroleate; PEG-8 stearate; PEG-40 stearate; PEG-50 stearate; PEG-100 stearate; sorbitan laurate; sorbitan palmitate; sorbitan stearate; sorbitan tristearate; sorbitan oleate; sorbitan trioleate; polysorbate 20; polysorbate 21; polysorbate 40; polysorbate 60; polysorbate 61; polysorbate 65; polysorbate 80; polysorbate 81; polysorbate 85; PEG-40 hydrogenated castor oil (also known as Emulsogen HCW-049); citric acid ester (also known as Citreni N12 Veg K), and combinations thereof.

### Adjunct Ingredients

The lotion composition, whether anhydrous or hydrophilic, may comprise any number of adjunct ingredients. Adjunct ingredients, which may include, for example, emollients, surfactants, rheological additives, and preservatives, may be functional ingredients or signal ingredients. Functional ingredients may contribute to the phase- or oxidative-stability of the composition, or may provide improved cleaning, skin health benefits, or aesthetic benefits, such as redness reduction. Signal ingredients may provide some marginal benefit, but may be included primarily for their role in communicating the benefits provided by a product. For example, a lotion may comprise a scent or perfume which is considered calming, soothing, refreshing, etc. Some exemplary adjunct ingredients are described in greater detail below, however, it will be readily understood that many other adjunct ingredients might be desirable and technically feasible in a wet-wipe product as described herein.

### Emollient

Emollients may (1) improve the glide of the substrate on the skin, by enhancing the lubrication and thus decreasing the abrasion of the skin, (2) hydrate the residues (for example, fecal residues or dried urine residues or menses), thus enhancing their removal from the skin, (3) hydrate the skin, thus reducing its dryness and irritation while improving its flexibility under the wiping movement, and (4) protect the skin from later irritation (for example, caused by the friction of an absorbent article) as the emollient is deposited onto the skin and remains at its surface as a thin protective layer.

Emollients may include silicone oils, functionalized silicone oils, hydrocarbon oils like mineral oil, petrolatum, vegetable oils like sunflower seed oil, fatty alcohols, fatty alcohol ethers, polysiloxanes, fatty acids, esters of monobasic and/or dibasic and/or tribasic and/or polybasic carboxylic acids with mono and polyhydric alcohols, polyoxyethylenes, polyoxypropylenes, mixtures of polyoxyethylene and polyoxypropylene ethers of fatty alcohols, and mixtures thereof. The emollients may be either saturated or unsaturated, have an aliphatic character and be straight or branched chained or contain alicyclic or aromatic rings. Mixtures of different emollients may be used.

In some embodiments, the oil material comprising the omega-6 fatty acid may serve as an emollient. As one example, the use of high oleic sunflower seed oil may function as both an emollient and a source of omega-6 fatty acids. Of course, it is also possible to employ variants of the same type of oil as an emollient and a source of omega-6 fatty acids. For example, a lotion and/or a coating may comprise both sunflower seed oil (as an emollient) and high oleic sunflower seed oil (as a source of omega-6 fatty acids).

A useful mixture of emollients is caprylic capric triglycerides in combination with Bis-PEG/PPG-16/16 PEG/PPG-16/16 dimethicone known as ABIL CARE™ 85 (available from Degussa Care Specialties of Hopewell, Va.),

### Essential Oils

A wet-wipe may optionally further comprise essential oil materials that help to connote the benefits provided by the wipe article. Such essential oil materials can be applied to the wipe article separate from the lotion composition or can be made part of the lotion composition. Non-limiting examples of suitable essential oil materials include Acorns gramineus, Anthemis nobilis, Artemisia dracunculus, Basil, Bergamot, Calamintha sylvatica, Caraway, Cedarwood, Chamomile, Cineol, Cinnamon, Cinnamon bark, Citrus aurantium, Clove, Cypress, Dill, Eucalyptus, Eugenol, Frankincense, Galangol, Geranium, Ginger, Hibiscus, Hop, Jasmine, Juniper, Laurus nobilis, Lavender, Lemon balm, Lemongrass, Lemon, Limonene, Linalool, Linalyl acetate, Lippia alba, Marjoram, Melissa, Myrrh, Neroli, Nutmeg, Passiflora, Patchouli, Peppermint, Pinene, Rose, Rosewood, Rosemary, Sage, Sandalwood, Spearmint, Sweet Fennel, Sweet Orange, Tea Tree, Thyme, Valerian, Ylang ylang, Zadoary, Hibiscus, or mixtures thereof. Oils associated with a harmonizing effect include Lavender, Neroli, and Ylang ylang.

### Surfactant

The surfactant can be an individual surfactant or a mixture of surfactants. The surfactant may be a polymeric surfactant or a non-polymeric surfactant. The surfactant may be employed as an emulsifier. The surfactant, when present, may be employed in an amount effective to emulsify the emollient, preservative, and any other non-water-soluble oils or ingredients that may be present in the composition. Alternatively, or in addition to, serving as an emulsifier of lotion components, the surfactant, if present, may help to emulsify of soils on the skin to be cleaned by the wipe, to lift the soils from the skin, and/or to keep the soils solubilized or suspended in the lotion until they can be lifted or wiped away with the wipe product.

The composition may include one or more surfactants. The surfactant or combinations of surfactants may be mild, which means that the surfactants provide sufficient cleansing or detersive benefits but do not overly dry or otherwise harm, damage, or irritate the skin.

A wide variety of surfactants are useful herein and include those selected from the group consisting of anionic surfactants, nonionic surfactants, cationic surfactants, amphoteric surfactants, zwitterionic surfactants, and mixtures thereof.

A wide variety of anionic surfactants may be useful herein. Non-limiting examples of anionic surfactants include those selected from the group consisting of sarcosinates, sulfates, sulfonates, isethionates, taurates, phosphates, lactylates, glutamates, and mixtures thereof. Amongst the isethionates, the alkoyl isethionates may be useful, and amongst the sulfates, the alkyl and alkyl ether sulfates may be useful. Other anionic materials useful herein include soaps (i.e., alkali metal or amine salts, e.g., sodium, potassium or triethanol amine salts) of fatty acids, typically having from about 8 to about 24 carbon atoms.

Nonionic surfactants useful herein include, but are not limited to, those selected from the group consisting of alkyl glucosides, alkyl polyglucosides, polyhydroxy fatty acid amides, alkoxylated fatty acid esters, alkoxylated fatty alcohol ethers, sorbitol esters like sorbitan monostearate, alkoxylated sorbitol esters like polysorbate 60, ethoxylated fatty alcohols like steareth-2 and steareth-20, sucrose esters, amine oxides, and mixtures thereof.

Suitable amphoteric or zwitterionic surfactants for use in the compositions herein include those which are known for use in hair care or other personal care cleansing. Amphoteric surfactants suitable for use in the present compositions are well known in the art and include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group such as carboxy, sulfonate, sulfate, phosphate, or phosphonate. Useful amphoteric surfactants include, but are not limited to, the group consisting of cocoamphoacetate, cocoamphodiacetate, lauroamphoacetate, lauroamphodiacetate, and mixtures thereof.

Zwitterionic surfactants suitable for use herein include those surfactants broadly described as derivatives of aliphatic quaternary ammonium, phosphonium, and sulfoniuin compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. Useful zwitterionic detersive surfactants are the betaines, amphoacetates and sulfobetaines, e.g., cocoamidopropylbetaine, sodiumlaurylamphoacetate and cocoamidopropylhydroxysultaine.

Loading the Lotion and/or Coating onto the Wipe Substrate

The wipe of the present invention can contain an effective amount of the lotion and/or coating composition. As used herein, the term “effective amount of a lotion (or coating) composition” refers to an amount of a particular lotion or coating composition which, when applied to a wipe, will be effective in transferring omega-6 fatty acid to the skin of the wearer. The effective amount of a lotion and/or coating composition will depend, to a large extent, on the particular lotion composition and/or coating used, the substrate used, and the primary intended purpose for the wipe (e.g., cleaning feces from a baby's bottom, as compared to cleaning food soils from hands, or makeup from the face). The amount of lotion and/or coating used may be described as a lotion load with respect to the weight of the “dry” or unloaded substrate (recognizing that the unloaded substrate may contain some moisture before lotion is applied). Suitable lotion loads for a wet-wipe product may range from 100 to 600%.

The lotion and/or coating composition can be applied to the surface of the wipe article. Suitable methods include spraying (including the use of a lotion bar), printing (e.g., flexographic printing), coating (e.g., gravure coating, slot coating), extrusion, slot coating, immersion or partial immersion (e.g., dipping), or combinations of these application techniques, e.g. spraying the lotion and/or coating composition on a rotating surface, such as a calender roll, that then transfers the composition to the outer surface of the topsheet. Lotion and/or coating compositions can be applied by printing methods, or continuous spray or extrusion, as is described in U.S. Pat. No. 5,968,025.

The manner of applying the lotion and/or coating composition to the surface of the wipe article may be such that the wipe article does not become saturated with the lotion and/or coating composition. In some embodiments, the wipe article may be saturated with the lotion and/or coating composition. In some embodiments, saturation of the wipe article is not required to obtain therapeutic and/or protective lotion and/or coating benefits. In some embodiments, the lotion and/or coating composition is applied primarily to the outer surface of the wipe article.

As mentioned above, the amount of lotion composition within each wet wipe may vary depending upon the type of material being used to provide the wet wipe or wipe-type product, the type of container being used to store the wet wipes, and the desired end use of the wet wipe. Generally, each wet wipe can contain from about 100 to about 600 weight percent, or from about 250 to about 450 weight percent lotion based on the unloaded weight of the wipe.

The amount of coating composition within each wipe may vary depending upon the type of material being used to provide the wipe, the type of container being used to store the wipes, and the desired end use of the wipe. Each wipe may contain coating from about 0.5% to about 250% by weight of the substrate, or from about 0.5% to about 100% by weight of the substrate, or from about 0.5% to about 25% by weight of the substrate.

The coating composition may be applied to the entire surface of the wipe article, or only portions thereof. The coating composition may, for example, be applied in a stripe aligned with and centered on the longitudinal centerline of the wipe. The coating composition can be applied in a plurality of stripes having uniform or non-uniform widths. Alternatively, the coating can be aligned with and centered in apposition to the longitudinal centerline.

In certain embodiments, the coating may be applied in a plurality of stripes parallel to the longitudinal axis of the wipe. This may allow for transfer of the coating to a broader area of the skin covered by an absorbent article (e.g., semi-occluded skin) and may contribute to improved fluid handling by the wipe.

The coating composition may be applied nonuniformly to the outer surface of the wipe article. By “nonuniform” is meant that the amount, pattern of distribution, etc., of the coating composition can vary over the wipe surface. For example, some portions of the treated surface of the wipe can have greater or lesser amounts of coating composition, including portions of the surface that do not have any coating composition on them. For example, the coating composition can be applied on one region of the wipe in the shape of a rectangle and/or a circle, and/or as mutliplicity of dots.

In some embodiments, a coating which is solid or semi-solid at room temperature may be applied to the outer surface of the wipe, such that it will transfer, as by pressure, body heat, or friction, to the skin when the skin is wiped. In some embodiments, a coating is applied in a discontinuous pattern to the outer surface of a wipe. Such a wipe may be further treated with an aqueous lotion. The aqueous lotion may permeate the substrate in the between the regions of the coating on the surface of the wipe. Alternately, an aqueous lotion may be applied to the wipe substrate without a coating.

The lotion composition can be applied to the surface at any point during assembly. For example, the lotion composition can be applied to the wipe article before it has been packaged.

The coating composition may be applied from a melt thereof to the wipe article. Since the coating composition will typically melt at significantly above ambient temperatures, it is usually applied as a heated coating. Typically, the coating composition is heated to a temperature in the range from about 35° C. to about 100° C., preferably from 40° C. to about 90° C., prior to being applied. Once the melted coating composition has been applied, it is allowed to cool and solidify to form a solidified coating or film on the surface of the wipe. The application process may be designed to aid in the cooling/set up of the coating, as by using air flow or cooled air to help solidify the coating. Crystallization agents, like paraffin waxes, can be added to the coating composition so it solidifies more quickly.

In certain embodiments, the wipe can be joined to a sanitary napkin, pantiliner, tampon, diaper, or other suitable disposable hygiene article, (for example, as described in detail in U.S. Pat. No. 5,569,230, U.S. Pat. No. 6,911,022 or WO 03/057122 A1). In another embodiment, the lotion and/or coating composition can be provided as a stand-alone product in the form of a product that can be applied to the wipe article or to the skin by hand (for example, as described in detail in U.S. Pat. No. 5,948,416). In another embodiment, the lotion and/or coating composition can be provided as a stand-alone product in the form of a spray or mousse that can be sprayed onto the wipe article or the skin by the wearer of an absorbent article (for example, as described in detail in U.S. Pat. No. 4,708,813).

### Method of Improving Skin Barrier Function of Semi-Occluded Skin

A method of improving skin bather function of semi-occluded skin may comprise the step of contacting said semi-occluded skin with a wipe article comprising an omega-6 fatty acid disposed on the wipe. Improvement in skin barrier function may be exhibited by improved skin lipid composition, improved skin moisturization, improved skin barrier formation, or the like. The wipe may comprise a lotion and/or coating composition, as described above.

A wipe article comprising omega-6 fatty acid, such as those described herein, may be used to improve the skin barrier function of semi-occluded skin.

The following are non—limiting examples of the present disclosure. In the Examples, “QS” refers herein to “quantum sufficit” and is a sufficient percentage of water added to the composition to bring the overall composition to 100%.

Wipes Comprising Only an Anhydrous Coating

The compositions exemplified hereinbelow in Tables 1 and 2 are examples of the coating compositions described above. The coating compositions are generally prepared by combining, by weight, the components in the first table below under heat until molten. Numbers below represent weight percents.

TABLE 1

| Component | Ex 1 | Ex 2 | Ex 3 | Ex 4 | Ex. 5 |
|---|---|---|---|---|---|
| SEFA* Cottonate | 38 | — | — | 9 | — |
| SEFA* Behenate | 12 | — | — | — | — |
| Petrolatum | 10 | 20 | 20 | 11 | 33 |
| Tribehenin | 5 | — | — | — | — |
| Cottonseed Oil | 10 | 60 | 6 | 30 | — |
| Lipex Omega 3/6 | — | — | 50 | | 40 |
| Lipex Omega Passiflora | — | — | — | 30 | — |
| C₁₀-C₃₀ Cholesterol/Lanosterol | 25 | — | — | — | 15 |
| Esters | | | | | |
| Synthetic Beeswax | — | 3 | 3 | 3 | 3 |
| Polyethylene Wax | — | 9 | 9 | 9 | 9 |
| Paraffin | — | 8 | 12 | 8 | — |

*Sucrose Ester Fatty Acid

TABLE 2

| Component | Ex. 8 | Ex. 7 | Ex. 8 | Ex. 9 | Ex. 10 | Ex. 11 |
|---|---|---|---|---|---|---|
| Petrolatum | 55.0 | 57.5 | 60.0 | 43.0 | 46.0 | — |
| Cetearyl Alcohol | 21.5 | 11.0 | 7.0 | 18.0 | 30.5 | — |
| Microcrystalline Wax | — | 10.5 | 8.0 | 17.0 | — | 5.0 |
| Paraffin Wax | — | — | 5.0 | 1.0 | — | |
| Beeswax | — | 2.5 | 1.5 | — | 1.0 | — |
| Carnauba Wax | 2.0 | — | — | 1.5 | — | 9.5 |
| High Oleic Canola Oil | — | — | 18.0 | 21.0 | — | — |
| High Oleic Sunflower Oil | 21.0 | 18.0 | — | — | 22.0 | 85.0 |
| Vitamin E | 0.5 | 0.5 | 0.5 | 0.5 | 0.5 | 0.5 |
| TOTAL | 100.00 | 100.00 | 100.00 | 100.00 | 100.00 | 100.00 |

Wet Wipe

The exemplary compositions in Tables 3 and 4 are representative of the lotion compositions described above. The lotion compositions of Examples 12 through 23 are contacted as described earlier with a substrate such as Fibrella 3160, a 58 grams/m² nonwoven comprising a blend of 40% viscose fibers and 60% polypropylene fibers as is available from Suominen of Tampere, Finland or any other substrate deemed suitable for use. Numbers represent weight percents.

TABLE 3

| Component | Ex. 12 | Ex. 13 | Ex. 14 | Ex. 15 | Ex. 16 | Ex. 17 | Ex. 18 |
|---|---|---|---|---|---|---|---|
| Water | Q.S | Q.S | Q.S | Q.S | Q.S | Q.S | Q.S |
| Disodium EDTA¹¹ | 0.100 | 0.100 | 0.100 | 0.100 | 0.100 | 0.100 | 0.100 |
| Iodopropynyl Butylcarbamate | 0.009 | 0.009 | 0.009 | 0.009 | 0.009 | 0.009 | 0.009 |
| (IPBC)¹ | | | | | | | |
| Benzyl alcohol² | 0.500 | 0.500 | 0.500 | 0.500 | 0.500 | 0.500 | 0.500 |
| Suttocide ® A (50% soln)³ | 0.150 | 0.150 | 0.150 | 0.150 | 0.150 | 0.150 | 0.150 |
| Phenoxyethanol¹¹ | 0.000 | 0.000 | 0.000 | 0.000 | 0.000 | 0.000 | 0.000 |
| Methyl Paraben¹¹ | 0.000 | 0.000 | 0.000 | 0.000 | 0.000 | 0.000 | 0.000 |
| Ethyl Paraben¹¹ | 0.000 | 0.000 | 0.000 | 0.000 | 0.000 | 0.000 | 0.000 |
| Propyl Paraben¹¹ | 0.000 | 0.000 | 0.000 | 0.000 | 0.000 | 0.000 | 0.000 |
| Xanthan Gum¹² | 0.180 | 0.180 | 0.180 | 0.180 | 0.180 | 0.180 | 0.180 |
| Sorbitan monostearate | 0.300 | 0.600 | 1.500 | 0.300 | 0.300 | 1.500 | 0.300 |
| Polysorbate 60 | 0.200 | 0.400 | 1.000 | 0.200 | 0.200 | 1.000 | 0.200 |
| Polysorbate 20¹⁰ | 0.030 | 0.030 | 0.030 | 0.030 | 0.030 | 0.030 | 0.030 |
| Abil Care 85⁴ | 0.100 | 0.300 | 0.100 | 0.100 | 0.100 | 0.100 | 0.100 |
| Monobasic Sodium Phosphate¹² | 0.000 | 0.000 | 0.000 | 0.000 | 0.000 | 0.000 | 0.000 |
| Citric Acid⁵ | 0.055 | 0.055 | 0.055 | 0.055 | 0.055 | 0.055 | 0.055 |
| Perfume | 0.070 | 0.070 | 0.070 | 0.070 | 0.070 | 0.070 | 0.070 |
| Dimethicone⁹ | 0.000 | 0.000 | 0.000 | 0.000 | 0.000 | 0.000 | 0.000 |
| Cyclomethicone⁹ | 0.000 | 0.000 | 0.000 | 0.000 | 0.000 | 0.000 | 0.000 |
| Lipex ® Omega Passiflora⁷ | 0.500 | 2.000 | 5.000 | 0.500 | 0.000 | 2.000 | 0.000 |
| Lipex ® Omega 3/6⁷ | 0.000 | 0.000 | 0.000 | 0.500 | 0.500 | 2.000 | 0.500 |
| High Oleic Canola Oil⁸ | 0.000 | 0.000 | 0.000 | 0.000 | 0.000 | 2.000 | 0.500 |
| Niacinamide⁵ | 2.000 | 2.000 | 0.000 | 0.000 | 2.000 | 0.000 | 2.000 |
| Glycerin⁶ | 1.000 | 0.000 | 1.000 | 0.000 | 0.500 | 0.000 | 3.000 |
| Panthenol⁵ | 0.500 | 0.500 | 0.500 | 0.000 | 0.500 | 0.000 | 0.500 |

TABLE 4

| Component | Ex. 19 | Ex. 20 | Ex. 21 | Ex. 22 | Ex. 23 |
|---|---|---|---|---|---|
| Water | Q.S | Q.S | Q.S | Q.S | Q.S |
| Disodium EDTA¹¹ | 0.100 | 0.100 | 0.100 | 0.100 | 0.100 |
| Iodopropynyl | 0.000 | 0.000 | 0.009 | 0.009 | 0.000 |
| Butylcarbamate (IPBC)¹ | | | | | |
| Benzyl alcohol² | 0.000 | 0.000 | 0.500 | 0.500 | 0.000 |
| Suttocide ® A | 0.000 | 0.000 | 0.150 | 0.150 | 0.000 |
| (50% soln)³ | | | | | |
| Phenoxyethanol¹¹ | 0.800 | 0.800 | 0.000 | 0.000 | 0.800 |
| Methyl Paraben¹¹ | 0.150 | 0.150 | 0.000 | 0.000 | 0.150 |
| Ethyl Paraben¹¹ | 0.050 | 0.050 | 0.000 | 0.000 | 0.050 |
| Propyl Paraben¹¹ | 0.050 | 0.050 | 0.000 | 0.000 | 0.050 |
| Xanthan Gum¹² | 0.180 | 0.180 | 0.000 | 0.000 | 0.000 |
| Sorbitan Monostearate | 0.300 | 1.500 | 1.500 | 0.300 | 1.200 |
| Polysorbate 60 | 0.200 | 1.000 | 1.000 | 0.200 | 0.800 |
| Polysorbate 20¹⁰ | 0.500 | 0.500 | 0.030 | 0.030 | 0.500 |
| Abil Care 85⁴ | 0.100 | 0.100 | 0.000 | 0.000 | 0.000 |
| Monobasic Sodium | 0.180 | 0.180 | 0.000 | 0.000 | 0.180 |
| Phosphate¹² | | | | | |
| Citric Acid⁵ | 0.000 | 0.000 | 0.055 | 0.055 | 0.000 |
| Perfume | 0.070 | 0.070 | 0.070 | 0.070 | 0.070 |
| Dimethicone⁹ | 0.000 | 0.100 | 0.200 | 0.200 | 0.100 |
| Cyclomethicone⁹ | 0.000 | 0.050 | 0.100 | 0.100 | 0.050 |
| Lipex ® Omega | 0.200 | 0.000 | 0.000 | 0.000 | 3.000 |
| Passiflora⁷ | | | | | |
| Lipex ® Omega 3/6⁷ | 0.200 | 4.000 | 5.000 | 0.000 | 0.000 |
| High Oleic Canola Oil⁸ | 0.000 | 0.000 | 0.000 | 0.500 | 0.000 |
| Niacinamide⁵ | 0.000 | 0.000 | 0.000 | 2.000 | 0.000 |
| Glycerin⁶ | 0.000 | 0.000 | 0.500 | 0.500 | 0.000 |
| Panthenol⁵ | 0.000 | 0.000 | 0.500 | 0.000 | 0.000 |

¹IPBC available from Clariant UK, Ltd.; Leeks, United Kingdom
²Benzyl Alcohol available from Symrise, Inc.; Teterboro, NJ
³Suttocide ® A (50% soln) available from International Specialty Products; Wayne, NJ
⁴Abil Care 85 available from Evonik Goldschmidt; Hopewell, VA
⁵Niacinamide, Panthenol, and Citric acid available from DSM Nutritional; Parsippany, NJ
⁶Glycerin available from Procter and Gamble; Cincinnati, OH
⁷Omega Oils available from Aarhus Karlshamn; Karlshamn, Sweden
⁸Oleic Canola Oil available from Clear Valley ® 75 High Oleic Canola Oil from Cargill
⁹Silicones Available from Dow Corning; Midland, MI
¹⁰Polysorbate available from BASF Inc.; Florham Park, NJ
¹¹Parabens and Phenoxyethanol available from DeWolf Chemical; East Providence, RI
¹²Monobasic Sodium Phosphate available from Charkit Chemical; Norwalk, CT

In preparing the solutions representative of examples 12-20 all materials are blended in suitable mixing vessels at room temperature using a propeller type mixer, Final blends are mixed using a homogenizer. Into one vessel (oil phase) is added Abil Care, 50% of the Lipex Omega Passiflora, Lipex Omega 3/6, and/or High Oleic Canola Oil, and preservative system. Into another vessel (water phase) are added the water phase ingredients of 90% water, non-ionic surfactants, glycerin, niaciamide, and panthenol. The water phase is then added to oil phase and the combined mixtures are homogenized. The xanthan gum predispered in the remaining 50% oil is added and the emulsion homogenized again. The mixture is neutralized to pH 5-5.5 and qs with water and homogenized. If an oil based fragrance is used, it is added to the oil phase step. Otherwise, the fragrance, if any, is added to the water phase step.

In preparing solutions representative of examples 21-23, all materials are blended in suitable mixing vessels at room temperature using a propeller type mixer. Final blends are mixed using a homogenizer. Into one vessel (water phase) are added 90% of the water, the emulsifiers, glycerin, niaciamide, and panthenol. Into another vessel (oil phase) are added dimethicone, cyclomethicone, Lipex Omega Passiflora, Lipex Omega 3/6, and/or High Oleic Canola Oil, and the preservative system. The water phase is then added to the oil phase and the combined mixtures are homogenized. The mixture is neutralized to pH 5-5.5 and qs with water and homogenized. If an oil based fragrance is used, it is added to the oil phase step. Otherwise, the fragrance, if any, is added to the water phase step.

Other suitable lotion compositions which could be adapted to include an omega-6 fatty acid as described herein include those compositions described in U.S. Publication Nos. 2005/0008680A1, 2005/0008681A1, 2006/0171971A1, 2007/0286893A1, and 2007/0286894A1, in WO Publication Nos. 2005/004834A1, 2005/007128A1, 2007/144814A1, and 2007/144819A1, and in U.S. Pat. Nos. 5,648,083 (issued Jul. 15, 1997 to Blieszner and Decker), 6,641,826 (issued Nov. 4, 2002 to Durden), 6,613,729 (issued Sep. 2, 2003 to Cole et al.), 6,673,358 (issued Jan. 6, 2004 to Cole et al.), and 7,666,827 (issued Feb. 23, 2010 to Marsh et al.).

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A wipe comprising a substrate and an oil material, the oil material comprising an omega-6 fatty acid having an OSI of at least about 10 hours.

2. The wipe of claim 1, further comprising a lotion, wherein the lotion comprises the oil material.

3. The wipe of claim 2, further comprising an emulsifier.

4. The wipe of claim 1, further comprising a coating, wherein the coating comprises the oil material.

5. The wipe of claim 4, further comprising a lotion.

6. The wipe of claim 5, wherein the lotion comprises an oil material, the oil material comprising an omega-6 fatty acid having an OSI of at least about 14 hours.

7. The wipe of claim 6, wherein the oil material in the coating is the same as the oil material in the lotion.

8. The wipe of claim 4, wherein the coating is applied non-uniformly to the wipe.

9. The wipe of claim 8, wherein the coating is applied in stripes separated from each other by stripes comprising no coating.

10. The wipe of claim 2, wherein the lotion comprises less than 3% by weight omega-3 fatty acids.

11. The wipe of claim 2, wherein the lotion comprises at least 50% by weight oleic fatty acids.

12. The wipe of claim 4, wherein the coating comprises less than 3% by weight omega-3 fatty acids.

13. The wipe of claim 4, wherein the coating comprises at least 50% by weight oleic fatty acids.

14. The wipe of claim 4, wherein the coating comprises at least 5% by weight of a wax.

15. The wipe of claim 14, wherein the coating has a melt temperature or melt range greater than or equal to 30° C.

16. A method of improving skin barrier function of semi-occluded skin, said method comprising the step of contacting the semi-occluded skin with a wipe according to claim 1.

17. The wipe of claim 2, wherein the lotion composition comprises from about 0.003% to about 35%, by weight of the omega-6 fatty acid, and from about 0.01% to about 56% of oleic acid.

18. The wipe of claim 1, wherein the oil material is selected from the group consisting of a mixture of passiflora incarnata seed oil and vegetable oil, a mixture of camelina sativa seed oil and vegetable oil, a mixture of evening primrose oil and vegetable oil, high oleic canola oil, and mixtures thereof.

19. The wipe of claim 18, wherein said oil material has an oil stability index value of at least about 14 hours.

20. The wipe of claim 1, wherein the wipe further comprises an anti-oxidant material selected from the group consisting of α-tocopherol, β-tocopherol, γ-tocopherol, δ-tocopherol, α-tocotrienol, γ-tocotrienol, δ-tocotrienol, sesamolin, sesamin, catechin, and mixtures thereof.
